(19) United States
(12) Patent Application Publication
(10) Pub. No.: US 2022/0102011 A1
(43) Pub. Date: Mar. 31, 2022

(54) PREDICTIVE DATA ANALYSIS TECHNIQUES FOR CROSS-TREND DISEASE TRANSMISSION DETECTION

(71) Applicant: UnitedHealth Group Incorporated, Minnetonka, MN (US)

(72) Inventors: Alison R. Stroh, Minnetonka, MN (US); Elya Papoyan, Glendale, CA (US); Terence D. Wedam, Klamath Falls, OR (US); Robert J. Nation, Rancho Palos Verdes, CA (US); Stephen R. Dion, Reading, MA (US); Salman Ahmed, Hyderabad (IN)

(21) Appl. No.: 17/363,687

(22) Filed: Jun. 30, 2021

Related U.S. Application Data
(60) Provisional application No. 63/085,236, filed on Sep. 30, 2020.

(51) Int. Cl. G16H 50/80; G06N 5/04

(52) U.S. Cl. CPC G16H 50/80; G06N 5/04

(57) ABSTRACT

Various embodiments of the present invention provide methods, apparatus, systems, computing devices, computing entities, and/or the like for performing predictive data analysis using cross-trend disease transmission detection. Certain embodiments of the present invention utilize systems, methods, and computer program products that perform cross-trend disease transmission predictive data analysis by utilizing a predictive data analysis user interface configured to depict interactions between a case-based transmission rate trend user interface element, a superior domain death-based transmission rate trend user interface element, and an inferior domain death-based transmission rate user interface using case-based confidence interval user interface elements, superior domain death-based transmission rate user interface elements, and inferior domain death-based transmission rate user interface elements.

### CROSS-REFERENCES TO RELATED APPLICATION(S)

The present application claims priority to U.S. Provisional Patent Application No. 63/085,236 (filed Sep. 30, 2020), which is incorporated herein by reference in its entirety.

### BACKGROUND

Various embodiments of the present invention address technical challenges related to performing predictive data analysis and address the efficiency and reliability shortcomings of existing predictive data analysis solutions.

### BRIEF SUMMARY

In general, embodiments of the present invention provide methods, apparatus, systems, computing devices, computing entities, and/or the like for performing predictive data analysis using cross-trend disease transmission detection. Certain embodiments of the present invention utilize systems, methods, and computer program products that perform cross-trend disease transmission predictive data analysis by utilizing a predictive data analysis user interface configured to depict interactions between a case-based transmission rate trend user interface element, a superior domain death-based transmission rate trend user interface element, and an inferior domain death-based transmission rate user interface using case-based confidence interval user interface elements, superior domain death-based transmission rate user interface elements, and inferior domain death-based transmission rate user interface elements.

In accordance with one aspect, a method is provided. In one embodiment, the method comprises: identifying a case-based transmission rate trend for a superior geographic domain, wherein the case-based transmission is associated with a total time period comprising a prior time period and a current time period; identifying a superior domain death-based transmission rate trend for the superior geographic domain, wherein the superior domain death-based transmission rate trend is associated with the prior time period; identifying an inferior domain death-based transmission rate trend for an inferior geographic domain associated with the superior geographic domain, wherein the inferior domain death-based transmission rate trend is associated with the prior time period; and causing the presentation of a predictive data analysis user interface, wherein the predictive data analysis user interface is configured to: (i) display a case-based transmission rate trend user interface element corresponding to the case-based transmission rate trend, a superior domain death-based transmission rate trend user interface element corresponding to the superior domain death-based transmission rate trend, and an inferior domain death-based transmission rate trend user interface element corresponding to the inferior domain death-based transmission rate trend, (ii) upon user interaction with a total time period segment of the case-based transmission rate trend user interface element that is associated with a total time period timestamp in the total time period, display a case-based confidence interval user interface element that is configured to describe a plurality of case-based transmission rate trend confidence interval values for the total time period timestamp, and (iii) upon user interaction with a prior time period segment of the case-based transmission rate user interface element that corresponds to a prior time period timestamp in the prior time period, display: (a) a superior domain death-based transmission rate user interface element that is configured to describe a superior domain death-based transmission rate for the superior geographic domain and the prior time period timestamp, and (b) an inferior domain death-based transmission rate user interface element that is configured to describe an inferior domain death-based transmission rate for the inferior geographic domain and the prior time period timestamp.

In accordance with another aspect, a computer program product is provided. The computer program product may comprise at least one computer-readable storage medium having computer-readable program code portions stored therein, the computer-readable program code portions comprising executable portions configured to: identify a case-based transmission rate trend for a superior geographic domain, wherein the case-based transmission is associated with a total time period comprising a prior time period and a current time period; identify a superior domain death-based transmission rate trend for the superior geographic domain, wherein the superior domain death-based transmission rate trend is associated with the prior time period; identify an inferior domain death-based transmission rate trend for an inferior geographic domain associated with the superior geographic domain, wherein the inferior domain death-based transmission rate trend is associated with the prior time period; and cause the presentation of a predictive data analysis user interface, wherein the predictive data analysis user interface is configured to: (i) display a case-based transmission rate trend user interface element corresponding to the case-based transmission rate trend, a superior domain death-based transmission rate trend user interface element corresponding to the superior domain death-based transmission rate trend, and an inferior domain death-based transmission rate trend user interface element corresponding to the inferior domain death-based transmission rate trend, (ii) upon user interaction with a total time period segment of the case-based transmission rate trend user interface element that is associated with a total time period timestamp in the total time period, display a case-based confidence interval user interface element that is configured to describe a plurality of case-based transmission rate trend confidence interval values for the total time period timestamp, and (iii) upon user interaction with a prior time period segment of the case-based transmission rate user interface element that corresponds to a prior time period timestamp in the prior time period, display: (a) a superior domain death-based transmission rate user interface element that is configured to describe a superior domain death-based transmission rate for the superior geographic domain and the prior time period timestamp, and (b) an inferior domain death-based transmission rate user interface element that is configured to describe an inferior domain death-based transmission rate for the inferior geographic domain and the prior time period timestamp.

In accordance with yet another aspect, an apparatus comprising at least one processor and at least one memory including computer program code is provided. In one embodiment, the at least one memory and the computer program code may be configured to, with the processor, cause the apparatus to: identify a case-based transmission rate trend for a superior geographic domain, wherein the case-based transmission is associated with a total time period comprising a prior time period and a current time period; identify a superior domain death-based transmission rate trend for the superior geographic domain, wherein the superior domain death-based transmission rate trend is associated with the prior time period; identify an inferior domain death-based transmission rate trend for an inferior geographic domain associated with the superior geographic domain, wherein the inferior domain death-based transmission rate trend is associated with the prior time period; and cause the presentation of a predictive data analysis user interface, wherein the predictive data analysis user interface is configured to: (i) display a case-based transmission rate trend user interface element corresponding to the case-based transmission rate trend, a superior domain death-based transmission rate trend user interface element corresponding to the superior domain death-based transmission rate trend, and an inferior domain death-based transmission rate trend user interface element corresponding to the inferior domain death-based transmission rate trend, (ii) upon user interaction with a total time period segment of the case-based transmission rate trend user interface element that is associated with a total time period timestamp in the total time period, display a case-based confidence interval user interface element that is configured to describe a plurality of case-based transmission rate trend confidence interval values for the total time period timestamp, and (iii) upon user interaction with a prior time period segment of the case-based transmission rate user interface element that corresponds to a prior time period timestamp in the prior time period, display: (a) a superior domain death-based transmission rate user interface element that is configured to describe a superior domain death-based transmission rate for the superior geographic domain and the prior time period timestamp, and (b) an inferior domain death-based transmission rate user interface element that is configured to describe an inferior domain death-based transmission rate for the inferior geographic domain and the prior time period timestamp.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example client computing entity in accordance with some embodiments discussed herein.

FIG. 4 is a flowchart diagram of an example process for performing cross-trend disease transmission predictive data analysis in accordance with some embodiments discussed herein.

FIG. 5 is a flowchart diagram of an example process for performing data processing operations in accordance with some embodiments discussed herein.

FIG. 6 is a flowchart diagram of an example process generating a death-based transmission rate trend in accordance with some embodiments discussed herein.

FIGS. 7 provides an operational example of an age-based mortality rate distribution in accordance with some embodiments discussed herein.

FIG. 8 provides an operational example of a cross-timestamp mortality contribution distribution in accordance with some embodiments discussed herein.

FIGS. 9-11 provide operational examples of predictive data analysis user interfaces in accordance with some embodiments discussed herein.

### DETAILED DESCRIPTION

Various embodiments of the present invention now will be described more fully hereinafter with reference to the accompanying drawings, in which some, but not all, embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. OVERVIEW AND TECHNICAL IMPROVEMENTS

Various embodiments of the present invention address technical challenges related to improving efficiency and reliability of performing predictive data analysis with respect to spread. In many contexts, disease spread predictive data analysis is complicated because while case-based data is current but is not always reliable, while death-based data is not current but more reliable. Another complicating factor is that data is not always available for inferior geographic domains such as counties. As a result of these complicating factors, many existing disease spread predictive data analysis solutions are unreliable and/or inefficient. In some cases, existing disease spread predictive data analysis solutions utilize enormous amounts of computational resources to perform complex predictive data analysis tasks.

Various embodiments of the present invention address the above-described technical challenges related to improving efficiency and reliability of performing disease spread predictive data analysis by providing techniques for inferring relationships across case-based transmission rate trends and death-based transmission rate trends. For example, in some embodiments, predictive insights may be generated using inferred relationships across a case-based transmission rate trend, a superior domain death-based transmission rate trend for a superior geographic domain, and an inferior domain death-based transmission rate trend for an inferior geographic domain. By utilizing the described techniques, the noted embodiments of the present invention draw predictive insights from case-based data as well as death-based to perform disease spread predictive data analysis tasks in a more reliable and efficient fashion. This in turn enables the noted embodiments to perform disease spread predictive data analysis more reliably and more efficiently relative to existing disease spread predictive data analysis solutions. This also enables the noted embodiments of the present invention to consume less computational resources compared to existing disease spread predictive data analysis when performing disease spread predictive data analysis tasks.

### II. DEFINITIONS

The term “disease-spread-related data object” may refer to a data entity that is configured to describe one or more data fields associated with spread of a particular disease/virus, such as one or more data fields associated with a number of active infection cases for the particular disease/virus in a defined geographic unit at a particular unit of time, one or more data fields associated with a number of reported deaths resulting from the particular disease/virus in a defined geographic unit at a particular unit of time, and/or the like. Examples of disease-spread-related data objects include data objects describing transmission rates, data objects describing death rates, data objects describing testing rates, data objects describing hospital admission rates, data objects describing social media activity indicative of disease spready for a respective disease/virus, and/or the like. In some embodiments, each disease-spread-related data object may be associated with a geographic domain as well as a timestamp. The geographic domain of the disease-spread-related data object may describe a geographic area whose corresponding disease-spread-related information are described by the disease-spread-related data object. Examples of geographic domains include superior geographic domains that each include a group of inferior geographic domains. For example, a superior geographic domain may include a state/province, while an inferior geographic domain may include an intra-state/intra-province (e.g., county, district, and/or the like) within the particular state/province. The timestamp of the disease-spread-related data object may describe a unit of time (e.g., an hour, a day, a week) whose corresponding disease-spread-related information are described by the disease-spread-related data object.

The term “transmission rate” may refer to a data entity that is configured to describe a value that describes an expected/inferred/estimated transmissibility rate of a disease/virus across a defined geographic domain at a particular unit of time defined by a timestamp. Examples of transmission rates include case-based transmission rates that are determined based on data describing a measure of a number of active infection cases (e.g., data describing hospitalizations, data describing positive test results, and/or the like) of a particular disease/virus, as well as death-based transmission rates that are determined based on data describing a measure of a number of deaths from a particular disease/virus. An example of a case-based transmission rate is an effective reproduction number (Rt), such as the Rt for a particular state at a particular unit of time.

The term “case-based transmission rate trend” may refer to a data entity that is configured to describe a group of case-based transmission rates for a superior geographic domain across a total time period that includes both a prior time period for which death-based transmission rates are available to the predictive data analysis computing entity and a current time period for which death-based transmission rates are not available to the predictive data analysis computing entity. For example, if death statistics are deemed to have a 30-day lag in suggesting reliable death-based transmission rates, then at Dec. 1, 2021: (i) the prior time period may end at Nov. 1, 2021, and (ii) the current time period may begin at Nov. 2, 2021 and end at Dec. 1, 2021. In this example, the case-based transmission rate trend may include a case-based transmission rate for a superior geographic domain (e.g., for a state) with respect to each defined timestamp (e.g., with respect to each day) within either the prior time period or the current time period. The time period that includes the prior time period and the current time period is referred to herein as the total time period.

The term “superior domain death-based transmission rate trend” may refer to a data entity that is configured to describe a group of death-based transmission rates for a superior geographic domain across a prior time period for which death-based transmission rates are available to the predictive data analysis computing entity. For example, if death statistics are deemed to have a 30-day lag in suggesting reliable death-based transmission rates, then at Dec. 1, 2021: (i) the prior time period may end at Nov. 1, 2021, and (ii) the current time period may begin at Nov. 2, 2021 and end at Dec. 1, 2021. In this example, the superior domain transmission rate trend may include a death-based transmission rate for a superior geographic domain (e.g., for a state) with respect to each defined timestamp (e.g., with respect to each day) within the defined period time period.

The term “inferior domain death-based transmission rate trend” may refer to a data entity that is configured to describe a group of death-based transmission rates for an inferior geographic domain across a prior time period for which death-based transmission rates are available to the predictive data analysis computing entity. For example, if death statistics are deemed to have a 30-day lag in suggesting reliable death-based transmission rates, then at Dec. 1, 2021: (i) the prior time period may end at Nov. 1, 2021, and (ii) the current time period may begin at Nov. 2, 2021 and end at Dec. 1, 2021. In this example, the inferior domain transmission rate trend may include a death-based transmission rate for an inferior geographic domain (e.g., for a county) with respect to each defined timestamp (e.g., with respect to each day) within the defined period time period.

The term “predictive data analysis user interface” may refer to a data entity that is configured to describe data related to the case-based transmission rate trend, the superior domain death-based transmission rate trend, and the inferior domain death-based transmission rate trend. In some embodiments, the predictive data analysis user interface is configured to display a case-based transmission rate trend user interface element corresponding to the case-based transmission rate trend, a superior domain death-based transmission rate trend user interface element corresponding to the superior domain death-based transmission rate trend, and an inferior domain death-based transmission rate trend user interface element corresponding to the inferior domain death-based transmission rate trend. In some embodiments, the predictive data analysis user interface is configured to: display a superior domain selection user interface element that is configured to enable selecting a selected superior geographic domain from a plurality of superior geographic domains, and display an inferior domain selection user interface element that is configure to enable selecting a selected inferior geographic domain from a plurality of inferior geographic domains selected for a selected superior geographic domain. In some embodiments, the predictive data analysis user interface is configured to: display a superior domain selection user interface element that is configured to enable selecting a selected superior geographic domain from a plurality of superior geographic domains, and display an inferior domain selection user interface element that is configure to enable selecting a selected inferior geographic domain from a plurality of inferior geographic domains selected for a selected superior geographic domain. In some embodiments, upon user interaction (e.g., user hovering over, user clicking on, user touching on, and/or the like) with a segment of the case-based transmission rate trend user interface element that corresponds to a total time period timestamp in the total time period, the predictive data analysis user interface is configured to display a case-based confidence interval user interface element that is configured to describe a plurality of case-based transmission rate trend confidence interval values for the total time period timestamp. In some embodiments, the case-based transmission rate trend describes a confidence interval for each case-based transmission rate associated with a total time period timestamp, where the confidence interval is associated with a plurality of case-based transmission rate trend confidence interval values (e.g., a case-based transmission rate confidence interval value corresponding to an 80% upper confidence interval, a case-based transmission rate confidence interval value corresponding to an 80% lower confidence interval, and/or the like). In some embodiments, the plurality of case-based transmission rate trend confidence interval values are configured to be displayed via the noted case-based confidence interval user interface elements. In some embodiments, upon user interface interaction with a segment of the case-based transmission rate trend user interface element that corresponds to a prior time period timestamp in the prior timestamp, the predictive data analysis user interface is configured to display: (a) a superior domain death-based transmission rate user interface element that is configured to describe a superior domain death-based transmission rate for the superior geographic domain and the prior time period timestamp, and (b) an inferior domain death-based transmission rate user interface element that is configured to describe an inferior domain death-based transmission rate for the inferior geographic domain and the prior time period timestamp.

The term “inferior domain confirmation determination” may refer to a data entity that is configured to describe a determination based on how much a prior subset of a case-based transmission rate trend that is associated with a prior time period corresponds to a superior domain death-based transmission rate trend. In some embodiments, to determine the inferior domain confirmation determination, a predictive data analysis computing entity first determines a measure of correspondence of a prior subset of a case-based transmission rate trend and a superior domain death-based transmission rate trend. Afterward, if the measure of correspondence satisfies (e.g., exceeds) a threshold, the predictive data analysis computing entity may determine that the inferior domain confirmation determination is a positive inferior domain confirmation determination. Moreover, if the measure of correspondence fails to satisfy (e.g., fail to exceed) a threshold, the predictive data analysis computing entity may determine that the inferior domain confirmation determination is a negative inferior domain confirmation determination. In some embodiments, in response to determining that the death-based confirmation determination describes the positive determination, the predictive data analysis computing entity determines an inferior domain transmission rate determination based on the inferior domain death-based transmission rate. In some embodiments, in response to determining that the death-based confirmation determination describes the positive determination, the predictive data analysis computing entity causes performance of one or more resource allocation actions with respect to the inferior geographic domain based on the inferior domain transmission rate determination.

### III. COMPUTER PROGRAM PRODUCTS, METHODS, AND COMPUTING ENTITIES

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. EXEMPLARY SYSTEM ARCHITECTURE

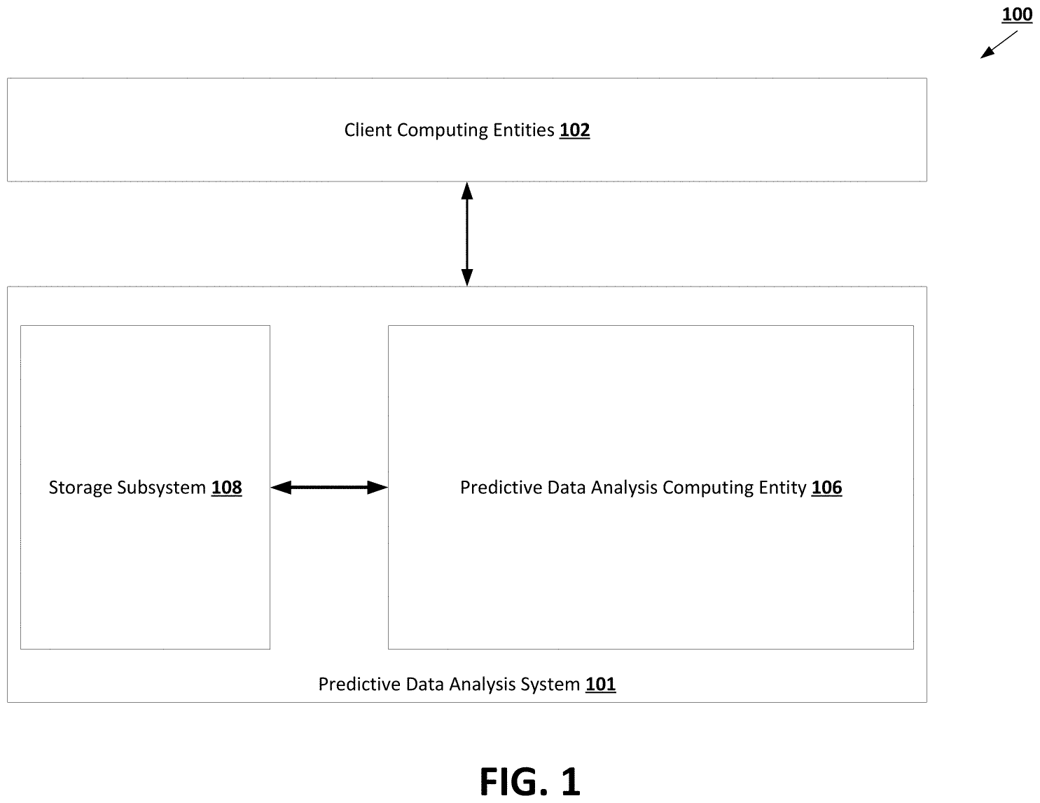

FIG. 1 is a schematic diagram of an example architecture 100 for performing predictive data analysis. The architecture 100 includes a predictive data analysis system 101 configured to receive predictive data analysis requests from client computing entities 102, process the predictive data analysis requests to generate predictions, provide the generated predictions to the client computing entities 102, and automatically perform prediction-based actions based at least in part on the generated predictions. An example of a prediction-based action that can be performed using the predictive data analysis system 101 is a request for generating a predictive data analysis user interface that enables performing cross-trend predictive data analysis. Another An example of a prediction-based action that can be performed using the predictive data analysis system 101 is a request for displaying a resource utilization recommendation based on an inferior domain death-based transmission rate trend that is deemed to have been confirmed.

In some embodiments, predictive data analysis system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive predictive data analysis requests from one or more client computing entities 102, process the predictive data analysis requests to generate predictions corresponding to the predictive data analysis requests, provide the generated predictions to the client computing entities 102, and automatically perform prediction-based actions based at least in part on the generated predictions.

The storage subsystem 108 may be configured to store input data used by the predictive data analysis computing entity 106 to perform predictive data analysis as well as model definition data used by the predictive data analysis computing entity 106 to perform various predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

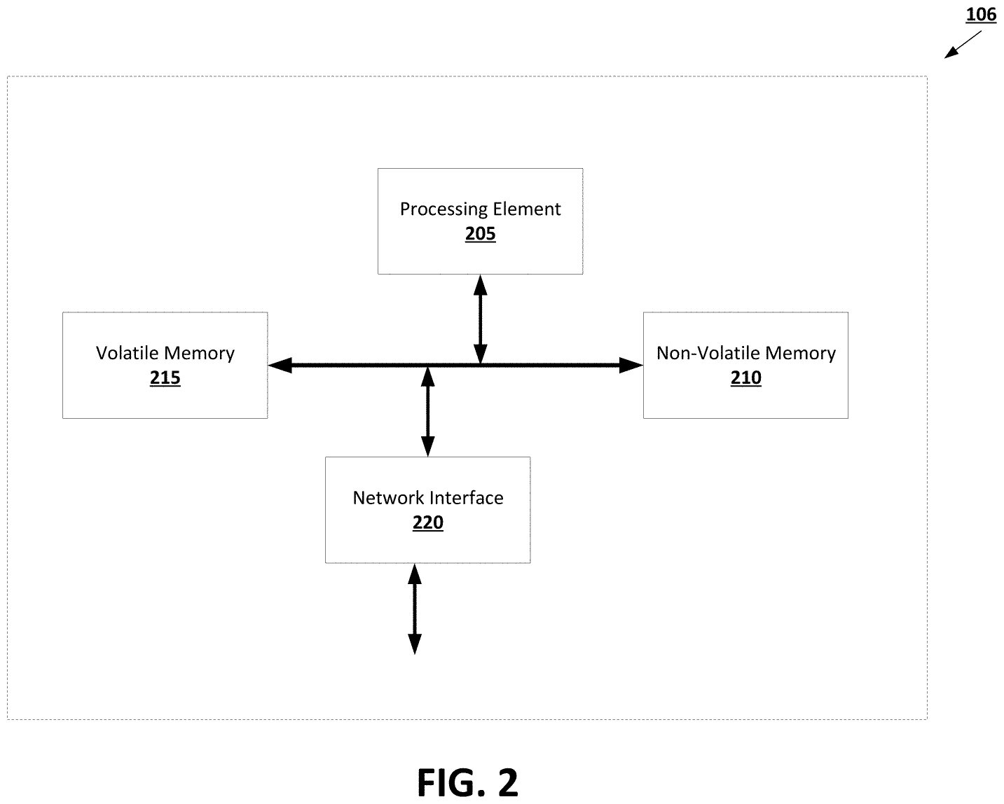

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the predictive data analysis computing entity 106 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

### Exemplary Client Computing Entity

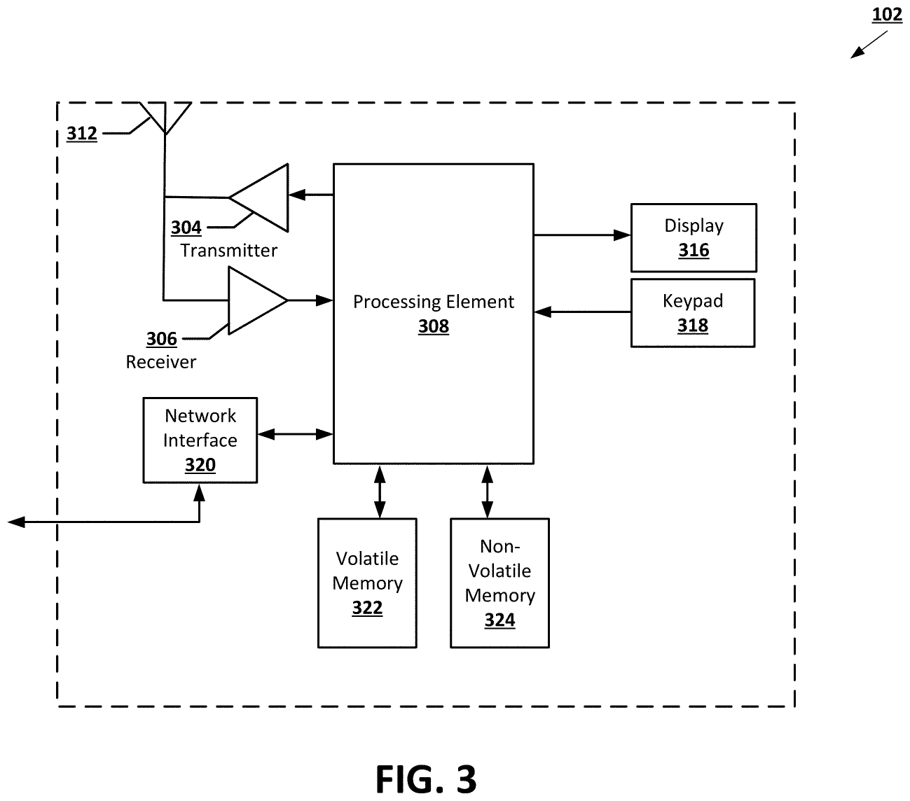

FIG. 3 provides an illustrative schematic representative of an client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the predictive data analysis computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the predictive data analysis computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the predictive data analysis computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the predictive data analysis computing entity 106 and/or various other computing entities.

In another embodiment, the client computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. EXEMPLARY SYSTEM OPERATIONS

Various embodiments of the present invention address the above-described technical challenges related to improving efficiency and reliability of performing disease spread predictive data analysis by providing techniques for inferring relationships across case-based transmission rate trends and death-based transmission rate trends. For example, in some embodiments, predictive insights may be generated using inferred relationships across a case-based transmission rate trend, a superior domain death-based transmission rate trend for a superior geographic domain, and an inferior domain death-based transmission rate trend for an inferior geographic domain. By utilizing the described techniques, the noted embodiments of the present invention draw predictive insights from case-based data as well as death-based to perform disease spread predictive data analysis tasks in a more reliable and efficient fashion. This in turn enables the noted embodiments to perform disease spread predictive data analysis more reliably and more efficiently relative to existing disease spread predictive data analysis solutions. This also enables the noted embodiments of the present invention to consume less computational resources compared to existing disease spread predictive data analysis when performing disease spread predictive data analysis tasks.

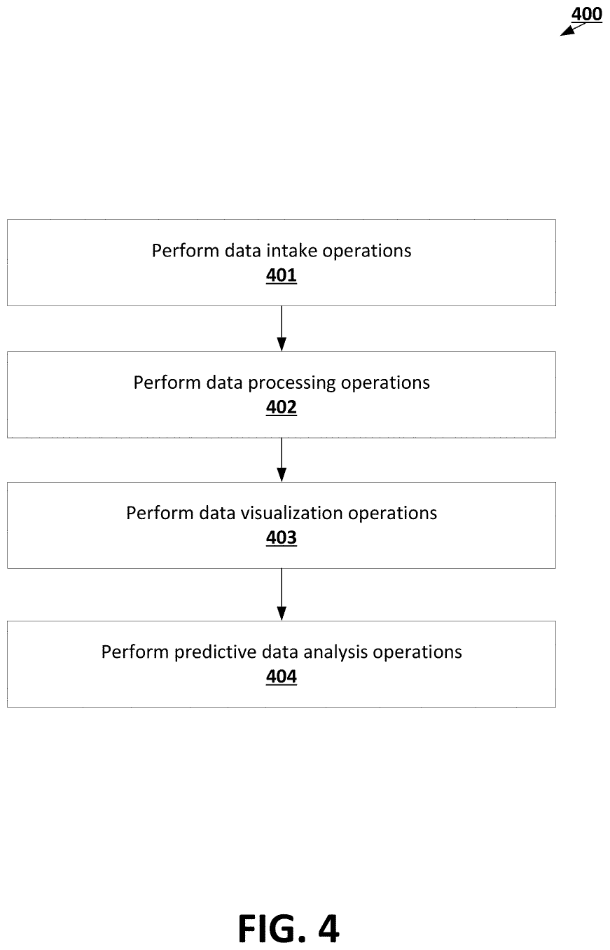

FIG. 4 is a flowchart diagram of an example process 400 for performing cross-trend disease transmission predictive data analysis. Via the various steps/operations of the process 400, the predictive data analysis computing entity 106 can enable using case-based transmission rate trends, superior domain death-based transmission rate trends, and inferior domain death-based transmission rate trends to perform effective and efficient determinations about current transmission rates across superior geographic domains (e.g., states) as well as inferior geographic domains (e.g., counties).

The process 400 begins at step/operation 401 when the predictive data analysis computing entity 106 performs one or more data intake operations to generate a group of disease-spread-related data objects. A disease-spread-related data object may describe one or more data fields associated with spread of a particular disease/virus, such as one or more data fields associated with a number of active infection cases for the particular disease/virus in a defined geographic unit at a particular unit of time, one or more data fields associated with a number of reported deaths resulting from the particular disease/virus in a defined geographic unit at a particular unit of time, and/or the like. Examples of disease-spread-related data objects include data objects describing transmission rates, data objects describing death rates, data objects describing testing rates, data objects describing hospital admission rates, data objects describing social media activity indicative of disease spready for a respective disease/virus, and/or the like. In some embodiments, each disease-spread-related data object may be associated with a geographic domain as well as a timestamp. The geographic domain of the disease-spread-related data object may describe a geographic area whose corresponding disease-spread-related information are described by the disease-spread-related data object. Examples of geographic domains include superior geographic domains that each include a group of inferior geographic domains. For example, a superior geographic domain may include a state/province, while an inferior geographic domain may include an intra-state/intra-province (e.g., county, district, and/or the like) within the particular state/province. The timestamp of the disease-spread-related data object may describe a unit of time (e.g., an hour, a day, a week) whose corresponding disease-spread-related information are described by the disease-spread-related data object.

Performing data intake may include retrieving data from one or more client computing entities, such as from the rt.live server and/or from a social media network server. In some embodiments, performing data intake includes: (i) intaking external public data into the predictive data analysis system 101 (e.g., determining when external data has been updated, pulling in data updates from external sources, pushing notification of data updates to interested parties, and/or the like), and (ii) cleansing of intake data for use in data processing (e.g., cleaning county/Federal Information Processing Standard Publication (FIPS) data to standardize the intake data, joining county-based data into an internal master geography dataset, standardizing formats for dates associated with data fields, and/or the like).

At step/operation 402, the predictive data analysis computing entity 106 performs one or more data processing operations on the group of disease-spread-related data objects to generate a group of transmission rates. A transmission rate may be a value that describes an expected/inferred/estimated transmissibility rate of a disease/virus across a defined geographic domain at a particular unit of time defined by a timestamp. Examples of transmission rates include case-based transmission rates that are determined based on data describing a measure of a number of active infection cases (e.g., data describing hospitalizations, data describing positive test results, and/or the like) of a particular disease/virus, as well as death-based transmission rates that are determined based on data describing a measure of a number of deaths from a particular disease/virus. An example of a case-based transmission rate is an effective reproduction number (Rt), such as the Rt for a particular state at a particular unit of time.

In some embodiments, the group of transmission rates are described by at least three data objects: (i) a case-based transmission rate trend, (ii) a superior domain death-based transmission rate trend, and (iii) an inferior domain death-based transmission rate trend. A case-based transmission rate trend may describe a group of case-based transmission rates for a superior geographic domain across a total time period that includes both a prior time period for which death-based transmission rates are available to the predictive data analysis computing entity and a current time period for which death-based transmission rates are not available to the predictive data analysis computing entity. For example, if death statistics are deemed to have a 30-day lag in suggesting reliable death-based transmission rates, then at Dec. 1, 2021: (i) the prior time period may end at Nov. 1, 2021, and (ii) the current time period may begin at Nov. 2, 2021 and end at Dec. 1, 2021. In this example, the case-based transmission rate trend may include a case-based transmission rate for a superior geographic domain (e.g., for a state) with respect to each defined timestamp (e.g., with respect to each day) within either the prior time period or the current time period. The time period that includes the prior time period and the current time period is referred to herein as the total time period.

A superior domain death-based transmission rate trend may describe a group of death-based transmission rates for a superior geographic domain across a prior time period for which death-based transmission rates are available to the predictive data analysis computing entity. For example, if death statistics are deemed to have a 30-day lag in suggesting reliable death-based transmission rates, then at Dec. 1, 2021: (i) the prior time period may end at Nov. 1, 2021, and (ii) the current time period may begin at Nov. 2, 2021 and end at Dec. 1, 2021. In this example, the superior domain transmission rate trend may include a death-based transmission rate for a superior geographic domain (e.g., for a state) with respect to each defined timestamp (e.g., with respect to each day) within the defined period time period.

An inferior domain death-based transmission rate trend may describe a group of death-based transmission rates for an inferior geographic domain across a prior time period for which death-based transmission rates are available to the predictive data analysis computing entity. For example, if death statistics are deemed to have a 30-day lag in suggesting reliable death-based transmission rates, then at Dec. 1, 2021: (i) the prior time period may end at Nov. 1, 2021, and (ii) the current time period may begin at Nov. 2, 2021 and end at Dec. 1, 2021. In this example, the inferior domain transmission rate trend may include a death-based transmission rate for an inferior geographic domain (e.g., for a county) with respect to each defined timestamp (e.g., with respect to each day) within the defined period time period.

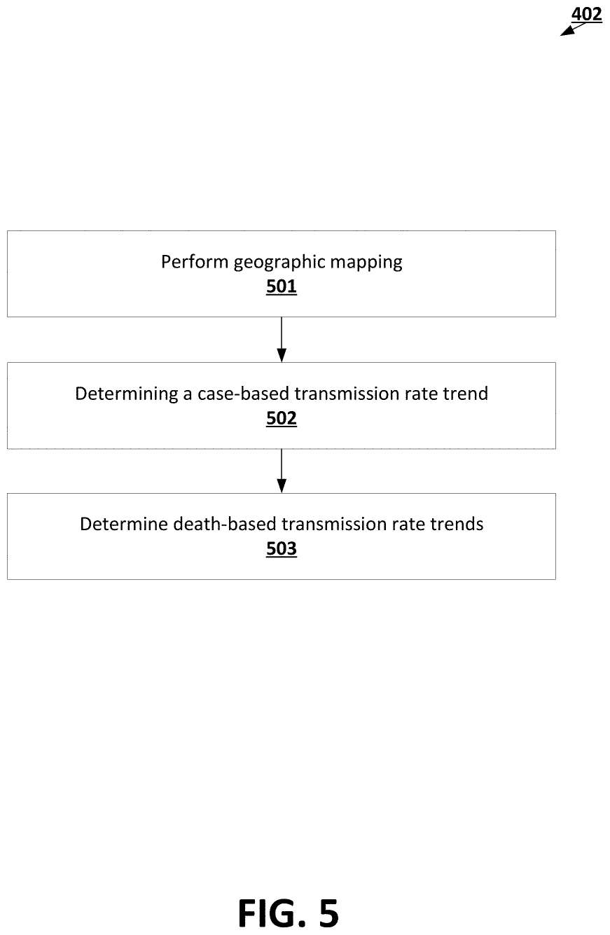

In some embodiments, step/operation 402 may be performed in accordance with the process that is depicted in FIG. 5. The process that is depicted in FIG. 5 begins at step/operation 501 when the predictive data analysis computing entity 106 performs geography mapping to associate each disease-spread-related data object of the group of disease-spread-related data objects to a geographic location in order to generate a group of location-mapped disease-spread-related data objects. This may entail mapping disease-spread-related data objects to metropolitan areas, government geographic units (e.g., states, provinces, counties, and/or the like), countries, continents, and/or the like. In some embodiments, performing geographic mapping entails: (i) determining the geographic location of disease-spread-related data objects, and (ii) mapping the determined geographic locations to an internal mapping associated with the predictive data analysis computing entity 106.

At step/operation 502, the predictive data analysis computing entity 106 determines a case-based transmission rate trend based on the group of location-mapped disease-spread-related data objects. As described above, the case-based transmission rate trend may describe a group of case-based transmission rates for a superior geographic domain across a total time period that includes both a prior time period for which death-based transmission rates are available to the predictive data analysis computing entity and a current time period for which death-based transmission rates are not available to the predictive data analysis computing entity.

At step/operation 503, the predictive data analysis computing entity 106 determines one or more death-based transmission rate trends based on the group of location-mapped disease-spread-related data objects. As described above, examples of death-based transmission rate trends include superior domain death-based transmission rate trends and inferior domain superior domain transmission rate trends. As further described above, a superior domain death-based transmission rate trend may describe a group of death-based transmission rates for a superior geographic domain across a prior time period for which death-based transmission rates are available to the predictive data analysis computing entity, while an inferior domain death-based transmission rate trend may describe a group of death-based transmission rates for an inferior geographic domain across a prior time period for which death-based transmission rates are available to the predictive data analysis computing entity.

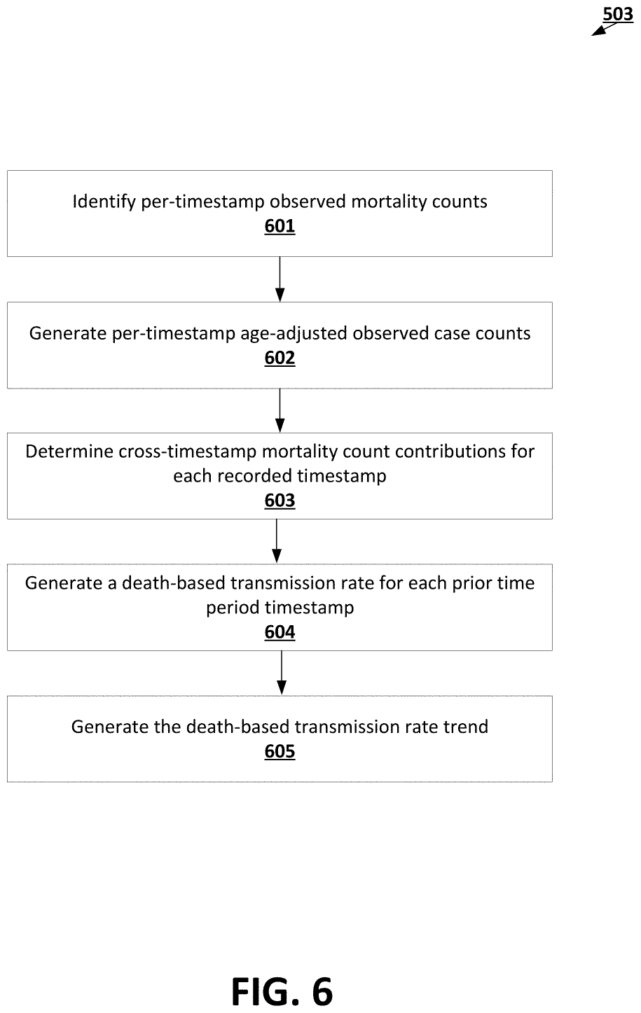

In some embodiments, step/operation 503 may be performed in accordance with the process that is depicted in FIG. 6 to generate a death-based transmission rate trend for a geographic domain (e.g., to generate a superior domain death-based transmission rate trend for a superior geographic domain such as a state and/or to generate an inferior domain death-based transmission rate trend for an inferior geographic domain such as a county). The process that is depicted in FIG. 6 begins at step/operation 601 when the predictive data analysis computing entity 106 identifies a group of per-timestamp observed mortality counts for the geographic domain, where each observed per-timestamp mortality count describes a recorded death count resulting from the particular disease/virus and for the geographic domain in a particular timestamp (i.e., a particular time unit) of a group of recorded timestamps associated with the death statistics data.

At step/operation 602, the predictive data analysis computing entity 106 determines a group of per-timestamp age-adjusted observed case counts for the geographic domain based on the group of per-timestamp observed mortality counts for the geographic domain and an age-adjusted mortality rate for the geographic domain, where each per-timestamp age-adjusted observed case count is associated with a recorded timestamp. In some embodiments, to generate a particular per-timestamp age-adjusted observed case count for a recorded timestamp, the predictive data analysis computing entity 106 performs an operation on the per-timestamp observed mortality count for the recorded timestamp and the age-adjusted mortality rate for the geographic domain (e.g., divides the per-timestamp observed mortality count for the recorded timestamp by the age-adjusted mortality rate for the geographic domain).

The age-adjusted mortality rate for a geographic domain may describe an inferred rate of death from a particular disease/virus in a geographic domain that is determined based on: (i) an age distribution of the geographic domain that describes a ratio of the population of the geographic domain that is deemed to belong to a particular age group of a group of age groups, and (ii) an age-based mortality rate distribution of the particular disease/virus that describes a likelihood that an individual infected with the particular/disease virus dies from the particular disease/virus given the age group of the individual.

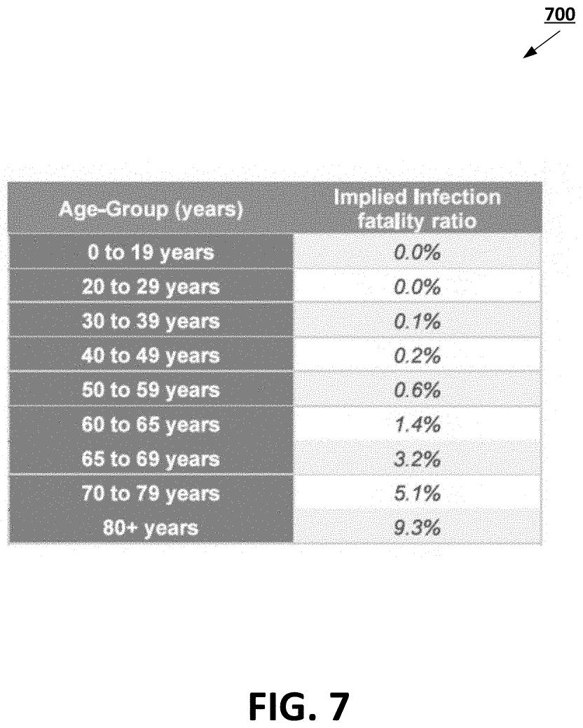

An operational example of an age-based mortality rate distribution 700 is depicted in FIG. 7. The age-based mortality rate distribution 700 can be used to determine the age-adjusted mortality rate for a geographic domain. For example, if 10 percent of a particular geographic domain are in the first listed age group, 10 percent of the particular geographic domain are in the second listed age group, 10 percent of the particular geographic domain are in the third listed age group, 10 percent of the particular geographic domain are in the fourth listed age group, 10 percent of the particular geographic domain are in the fifth listed age group, 10 percent of the particular geographic domain are in the sixth listed age group, 10 percent of the particular geographic domain are in the seventh listed age group, 10 percent of the particular geographic domain are in the eighth listed age group, and 20 percent of the particular geographic domain are in the ninth listed age group, then the age-adjusted mortality rate for the particular geographic may be determined using the following set of operations: (0.1*0.0%)+(0.1*0.0%)+(0.1*0.1%)+(0.1*0.2%)+(0.1*0.6%)+(0.1*1.4%)+(0.1*3.2%)+(0.1*5.1%)+(0.1*9.3%).

At step/operation 603, the predictive data analysis computing entity 106 determines, for each recorded timestamp of the group of recorded timestamps, a group of cross-timestamp mortality count contributions, where the group of cross-timestamp mortality count contributions includes a cross-timestamp mortality count contribution for each previous timestamp of a predefined number of previous timestamps before the recorded timestamp. For example, for each particular day of a group of recorded days associated with the group of per-timestamp age-adjusted observed case counts, the predictive data analysis computing entity 106 may determine a group of cross-timestamp mortality count contributions that include a cross-timestamp mortality count contribution for the particular day with respect to each previous day of a predefined number of previous days (e.g., 41 previous days) before the particular days.

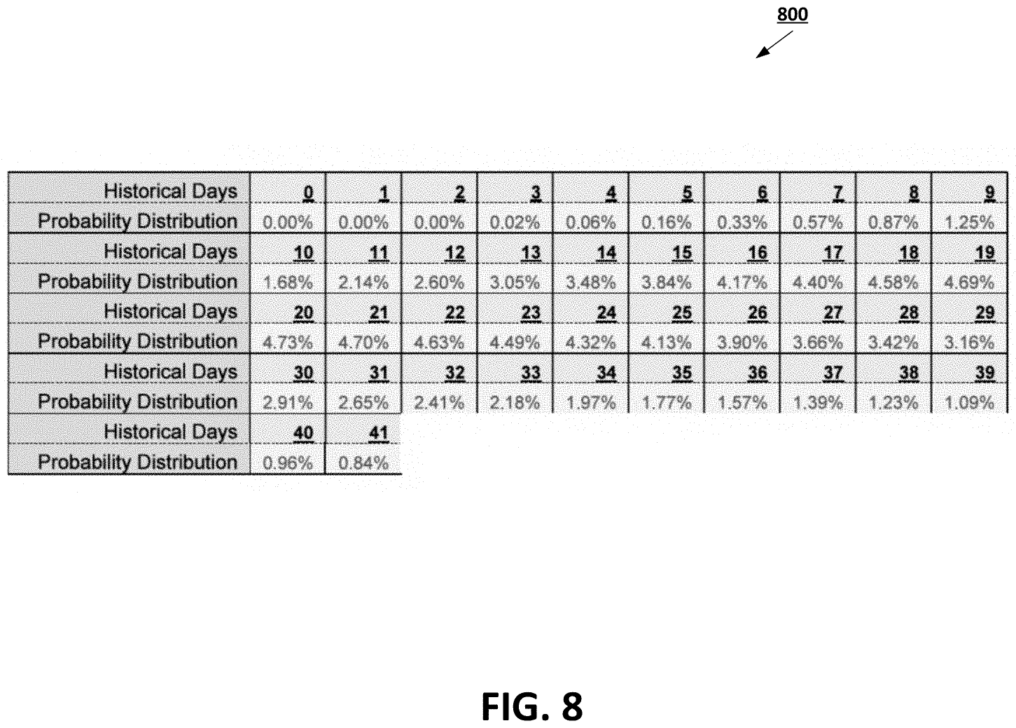

In some embodiments, determining the group of cross-timestamp mortality count contributions for a particular recorded timestamp may be performed with respect to a cross-timestamp mortality contribution distribution that describes, for each previous timestamp of a predefined number of previous timestamps before a particular timestamp, the likelihood that a unit per-timestamp age-adjusted observed case count in the particular timestamp implies a unit of death count within the previous timestamp. An operational example of a cross-timestamp mortality contribution distribution 800 is depicted in FIG. 8. As depicted in FIG. 8, in accordance with the cross-timestamp mortality contribution distribution 800, the likelihood that a unit per-timestamp age-adjusted observed case count in a particular timestamp implies a unit of death count in the 0^{th} day before the particular timestamp is 0.00%, while the likelihood that a unit per-timestamp age-adjusted observed case count in a particular timestamp implies a unit of death count in the 31^{th} day before the particular timestamp is 2.65%. Using the cross-timestamp mortality contribution distribution 800 and an exemplary per-timestamp age-adjusted observed case count of n for a particular day, the predictive data analysis computing entity 106 may determine that the cross-timestamp mortality count contribution for the particular day and the 31^{st} day before the particular day is 2.65%*n.

At step/operation 604, the predictive data analysis computing entity 106 combines, for each prior time period timestamp of a group of prior time period timestamps in the prior time period associated with the death-based transmission rate trend, each cross-timestamp mortality count contribution for the prior time period to generate the death-based transmission rate for the prior time period. In some embodiments, performing this step/operation may entail combining, for each prior time period timestamp of a group of prior time period timestamps, each cross-timestamp mortality count contribution that is determined for the prior time period timestamp based on a per-timestamp age-adjusted mortality count for a subsequent time period of a predefined number of subsequent time periods after the prior time period timestamp. For example, in the exemplary embodiment in which cross-timestamp mortality count contributions are determined for previous timestamps up to 41 previous timestamps, then, to determine death-based transmission rate for a particular day, the predictive data analysis computing entity 106 may combine each cross-timestamp mortality count contribution determined for that particular day using cross-timestamp mortality count contributions generated based on per-timestamp age-adjusted observed case counts for 41 subsequent days after the particular day.

At step/operation 605, the predictive data analysis computing entity 106 combines each death-based transmission rate for a prior time period timestamp of a group of prior time period timestamps in the prior time period associated with the death-based transmission rate trend to generate the death-based transmission rate. If the death-based transmission rates are for a superior geographic domain, then the death-based transmission rate trend is a superior domain death-based transmission rate trend. However, if the death-based transmission rates are for an inferior geographic domain, then the death-based transmission rate trend is an inferior domain death-based transmission rate trend.

Returning to FIG. 4, at step/operation 403, the predictive data analysis computing entity 106 performs one or more data visualization operations by causing a client computing entity 102 to present a predictive data analysis user interface. The predictive data analysis user interface may be configured to display data related to the case-based transmission rate trend, the superior domain death-based transmission rate trend, and the inferior domain death-based transmission rate trend.

In some embodiments, the predictive data analysis user interface is configured to display a case-based transmission rate trend user interface element corresponding to the case-based transmission rate trend, a superior domain death-based transmission rate trend user interface element corresponding to the superior domain death-based transmission rate trend, and an inferior domain death-based transmission rate trend user interface element corresponding to the inferior domain death-based transmission rate trend. In some embodiments, the predictive data analysis user interface is configured to: display a superior domain selection user interface element that is configured to enable selecting a selected superior geographic domain from a plurality of superior geographic domains, and display an inferior domain selection user interface element that is configure to enable selecting a selected inferior geographic domain from a plurality of inferior geographic domains selected for a selected superior geographic domain.

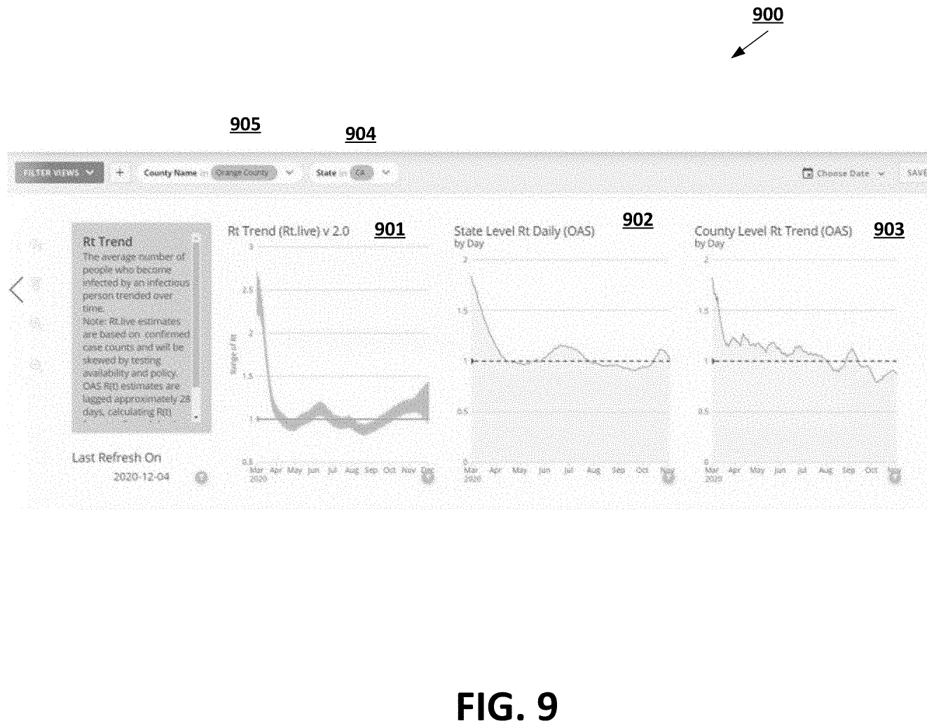

An operational example of a predictive data analysis user interface 900 is depicted in FIG. 9. As depicted in FIG. 9, the predictive data analysis user interface 900 includes the case-based transmission rate trend user interface element 901 which is a graph user interface element depicting information related to a case-based transmission rate trend, the superior domain death-based transmission rate trend user interface element 902 which is a graph user interface element depicting information related to a superior domain death-based transmission rate trend, and an inferior domain death-based transmission rate trend user interface element 903 which is a graph user interface element depicting information related to the inferior domain death-based transmission rate trend. As further depicted in FIG. 9, the predictive output user interface includes the superior domain selection user interface element 904 and the inferior domain selection user interface element 905.

In some embodiments, upon user interaction (e.g., user hovering over, user clicking on, user touching on, and/or the like) with a segment of the case-based transmission rate trend user interface element that corresponds to a total time period timestamp in the total time period, the predictive data analysis user interface is configured to display a case-based confidence interval user interface element that is configured to describe a plurality of case-based transmission rate trend confidence interval values for the total time period timestamp. In some embodiments, the case-based transmission rate trend describes a confidence interval for each case-based transmission rate associated with a total time period timestamp, where the confidence interval is associated with a plurality of case-based transmission rate trend confidence interval values (e.g., a case-based transmission rate confidence interval value corresponding to an 80% upper confidence interval, a case-based transmission rate confidence interval value corresponding to an 80% lower confidence interval, and/or the like). In some embodiments, the plurality of case-based transmission rate trend confidence interval values are configured to be displayed via the noted case-based confidence interval user interface elements.

In some embodiments, upon user interface interaction with a segment of the case-based transmission rate trend user interface element that corresponds to a prior time period timestamp in the prior timestamp, the predictive data analysis user interface is configured to display: (a) a superior domain death-based transmission rate user interface element that is configured to describe a superior domain death-based transmission rate for the superior geographic domain and the prior time period timestamp, and (b) an inferior domain death-based transmission rate user interface element that is configured to describe an inferior domain death-based transmission rate for the inferior geographic domain and the prior time period timestamp.

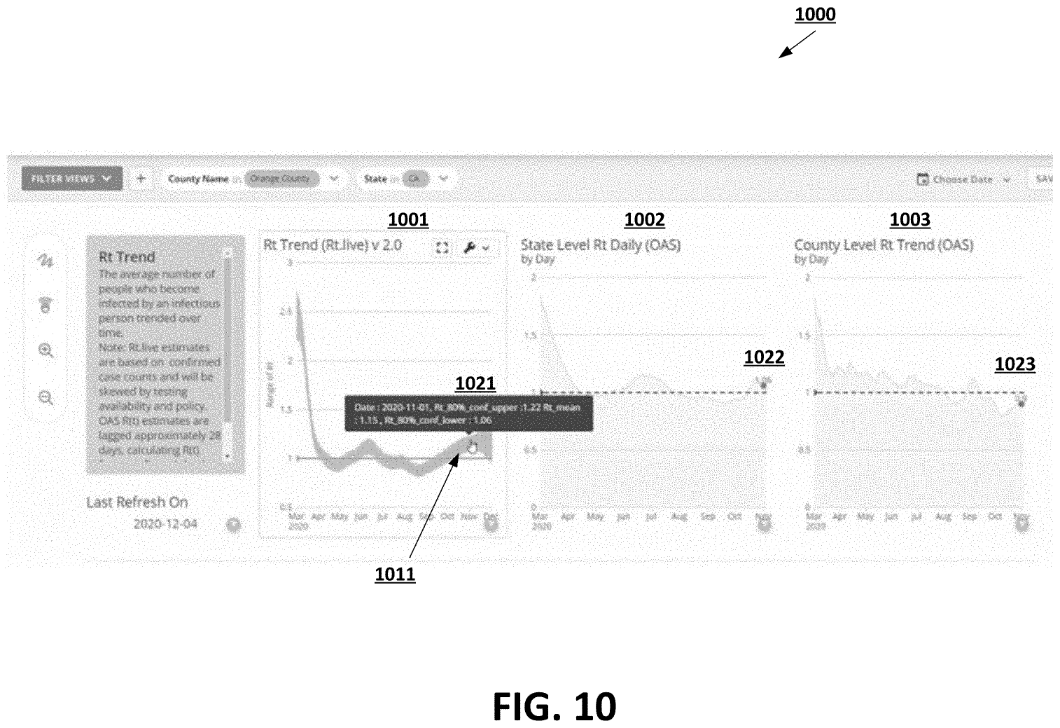

Another operational example of a predictive data analysis user interface 1000 is depicted in FIG. 10. As depicted in FIG. 10, the predictive data analysis user interface 1000 includes the case-based transmission rate trend user interface element 1001, the superior domain death-based transmission rate trend user interface element 1002, and the inferior domain death-based transmission user interface element 1003. As further depicted in FIG. 10, user interaction with a segment 1011 of the case-based transmission rate trend user interface element 1001 that is associated with a total time unit period (that is also a prior time unit period) causes: (i) display of the case-based confidence interval user interface element 1021 which is configured to display a case-based transmission rate confidence interval value corresponding to an 80% upper confidence interval and a case-based transmission rate confidence interval value corresponding to an 80% lower confidence interval, (ii) display of the corresponding superior domain death-based transmission rate user interface element 1022, and (iii) display of the corresponding inferior domain death-based transmission rate user interface element 1023.

In some embodiment, the predictive data analysis user interface may enable an end user to compare, for a selected timestamp, the plurality of case-based transmission rate trend confidence interval values for the selected timestamp as displayed via a case-based confidence interval user interface element with the superior domain death-based transmission rate for the selected timestamp as displayed by a superior domain death-based transmission rate trend user interface element. If the comparison demonstrates to the end user that the superior domain death-based transmission rate is within a sufficiently close range of a lower-bound case-based transmission rate trend confidence interval value of the plurality of case-based transmission rate trend confidence interval values, the end user may detect this as an indication that the inferior domain death-based transmission rates depicted by the inferior domain death-based transmission rate trend user interface element are reliable. As a result, the end user may use the inferior domain death-based transmission rates depicted by the inferior domain death-based transmission rate trend user interface element in decision-making, such as in resource allocation decision-making.

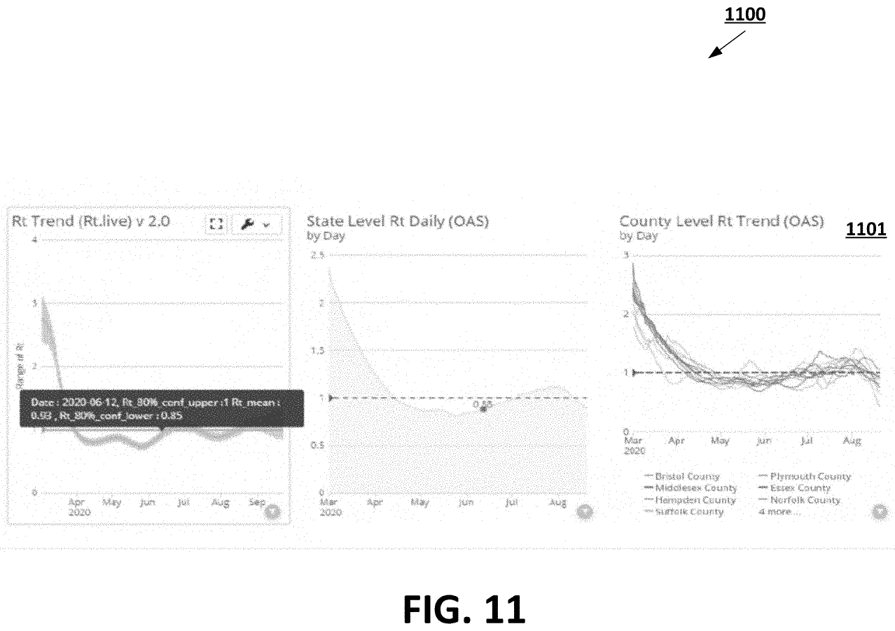

In some embodiments, when no inferior geographic domain is selected, the inferior domain death-based transmission rate trend user interface element of the predictive data analysis user interface is configured to depict a graph element corresponding to each inferior geographic domain of a plurality of inferior geographic domains of a selected superior geographic domain. An operational example of such an inferior domain death-based transmission rate trend user interface 1101 is depicted in the predictive data analysis user interface 1100 of FIG. 11.

Returning to FIG. 4, at step/operation 404, the predictive data analysis computing entity 106 optionally performs one or more predictive data analysis operations using the case-based transmission rate trend, the superior domain death-based transmission rate trend, and the inferior domain death-based transmission rate trend. For example, in some embodiments, the predictive data analysis computing entity 106 generates an inferior domain confirmation determination for the inferior domain death-based transmission rate trend for the case-based transmission rate trend based on comparing a prior subset of the case-based transmission rate trend that is associated with the prior time period and a superior domain death-based transmission rate trend. In some embodiments, in response to determining that the death-based confirmation determination describes the positive determination, the predictive data analysis computing entity 106 determines an inferior domain transmission rate determination based on the inferior domain death-based transmission rate. In some embodiments, in response to determining that the death-based confirmation determination describes the positive determination, the predictive data analysis computing entity 106 causes performance of one or more resource allocation actions with respect to the inferior geographic domain based on the inferior domain transmission rate determination.

An inferior domain confirmation determination may be a determination based on how much a prior subset of a case-based transmission rate trend that is associated with a prior time period corresponds to a superior domain death-based transmission rate trend. In some embodiments, to determine the inferior domain confirmation determination, a predictive data analysis computing entity first determines a measure of correspondence of a prior subset of a case-based transmission rate trend and a superior domain death-based transmission rate trend. Afterward, if the measure of correspondence satisfies (e.g., exceeds) a threshold, the predictive data analysis computing entity may determine that the inferior domain confirmation determination is a positive inferior domain confirmation determination. Moreover, if the measure of correspondence fails to satisfy (e.g., fail to exceed) a threshold, the predictive data analysis computing entity may determine that the inferior domain confirmation determination is a negative inferior domain confirmation determination. In some embodiments, in response to determining that the death-based confirmation determination describes the positive determination, the predictive data analysis computing entity determines an inferior domain transmission rate determination based on the inferior domain death-based transmission rate. In some embodiments, in response to determining that the death-based confirmation determination describes the positive determination, the predictive data analysis computing entity causes performance of one or more resource allocation actions with respect to the inferior geographic domain based on the inferior domain transmission rate determination.

### VI. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for performing cross-trend disease transmission predictive data analysis, the computer-implemented method comprising:
identifying, using one or more processors, a case-based transmission rate trend for a superior geographic domain, wherein the case-based transmission is associated with a total time period comprising a prior time period and a current time period;
identifying, using the one or more processors, a superior domain death-based transmission rate trend for the superior geographic domain, wherein the superior domain death-based transmission rate trend is associated with the prior time period;
identifying, using the one or more processors, an inferior domain death-based transmission rate trend for an inferior geographic domain associated with the superior geographic domain, wherein the inferior domain death-based transmission rate trend is associated with the prior time period; and
causing, using the one or more processors, the presentation of a predictive data analysis user interface, wherein the predictive data analysis user interface is configured to: (i) display a case-based transmission rate trend user interface element corresponding to the case-based transmission rate trend, a superior domain death-based transmission rate trend user interface element corresponding to the superior domain death-based transmission rate trend, and an inferior domain death-based transmission rate trend user interface element corresponding to the inferior domain death-based transmission rate trend, (ii) upon user interaction with a total time period segment of the case-based transmission rate trend user interface element that is associated with a total time period timestamp in the total time period, display a case-based confidence interval user interface element that is configured to describe a plurality of case-based transmission rate trend confidence interval values for the total time period timestamp, and (iii) upon user interaction with a prior time period segment of the case-based transmission rate user interface element that corresponds to a prior time period timestamp in the prior time period, display: (a) a superior domain death-based transmission rate user interface element that is configured to describe a superior domain death-based transmission rate for the superior geographic domain and the prior time period timestamp, and (b) an inferior domain death-based transmission rate user interface element that is configured to describe an inferior domain death-based transmission rate for the inferior geographic domain and the prior time period timestamp.

2. The computer-implemented method of claim 1, wherein the predictive data analysis user interface is further configured to:
display a superior domain selection user interface element that is configured to enable selecting a selected superior geographic domain from a plurality of superior geographic domains, and
display an inferior domain selection user interface element that is configure to enable selecting a selected inferior geographic domain from a plurality of inferior geographic domains selected for the selected superior geographic domain.

3. The computer-implemented method of claim 1, further comprising:
generating, using the one or more processors, an inferior domain confirmation determination for the inferior domain death-based transmission rate trend for the case-based transmission rate trend based on comparing a prior subset of the case-based transmission rate trend that is associated with the prior time period and the superior domain death-based transmission rate trend.

4. The computer-implemented method of claim 3, further comprising:
determining, using the one or more processors, whether the death-based confirmation determination describes a positive determination; and
in response to determining that the death-based confirmation determination describes the positive determination, determining, using the one or more processors, an inferior domain transmission rate determination based on the inferior domain death-based transmission rate.

5. The computer-implemented method of claim 4, further comprising:
in response to determining that the death-based confirmation determination describes the positive determination, causing, using the one or more processors, performance of one or more resource allocation actions with respect to the inferior geographic domain based on the inferior domain transmission rate determination.

6. The computer-implemented method of claim 1, wherein the case-based transmission rate trend describes a per-timestamp case-based transmission rate for each total time period timestamp of a plurality of total time period timestamps associated with the total time period.

7. The computer-implemented method of claim 1, wherein the superior domain death-based transmission rate trend describes a per-timestamp superior domain death-based transmission rate for each prior time period timestamp of a plurality of prior time period timestamps associated with the prior time period in relation to the superior geographic domain.

8. The computer-implemented method of claim 1, wherein the superior domain death-based transmission rate trend describes a per-timestamp superior domain death-based transmission rate for each prior time period timestamp of a plurality of prior time period timestamps associated with the prior time period in relation to the superior geographic domain.

9. An apparatus for performing cross-trend disease transmission predictive data analysis, the apparatus comprising at least one processor and at least one memory including program code, the at least one memory and the program code configured to, with the processor, cause the apparatus to at least:
identify a case-based transmission rate trend for a superior geographic domain, wherein the case-based transmission is associated with a total time period comprising a prior time period and a current time period;
identify a superior domain death-based transmission rate trend for the superior geographic domain, wherein the superior domain death-based transmission rate trend is associated with the prior time period;
identify an inferior domain death-based transmission rate trend for an inferior geographic domain associated with the superior geographic domain, wherein the inferior domain death-based transmission rate trend is associated with the prior time period; and
cause the presentation of a predictive data analysis user interface, wherein the predictive data analysis user interface is configured to: (i) display a case-based transmission rate trend user interface element corresponding to the case-based transmission rate trend, a superior domain death-based transmission rate trend user interface element corresponding to the superior domain death-based transmission rate trend, and an inferior domain death-based transmission rate trend user interface element corresponding to the inferior domain death-based transmission rate trend, (ii) upon user interaction with a total time period segment of the case-based transmission rate trend user interface element that is associated with a total time period timestamp in the total time period, display a case-based confidence interval user interface element that is configured to describe a plurality of case-based transmission rate trend confidence interval values for the total time period timestamp, and (iii) upon user interaction with a prior time period segment of the case-based transmission rate user interface element that corresponds to a prior time period timestamp in the prior time period, display: (a) a superior domain death-based transmission rate user interface element that is configured to describe a superior domain death-based transmission rate for the superior geographic domain and the prior time period timestamp, and (b) an inferior domain death-based transmission rate user interface element that is configured to describe an inferior domain death-based transmission rate for the inferior geographic domain and the prior time period timestamp.

10. The apparatus of claim 9, wherein the predictive data analysis user interface is further configured to:
display a superior domain selection user interface element that is configured to enable selecting a selected superior geographic domain from a plurality of superior geographic domains, and
display an inferior domain selection user interface element that is configure to enable selecting a selected inferior geographic domain from a plurality of inferior geographic domains selected for the selected superior geographic domain.

11. The apparatus of claim 9, wherein the at least one memory and the program code are configured to, with the processor, cause the apparatus to at least:
generate an inferior domain confirmation determination for the inferior domain death-based transmission rate trend for the case-based transmission rate trend based on comparing a prior subset of the case-based transmission rate trend that is associated with the prior time period and the superior domain death-based transmission rate trend.

12. The apparatus of claim 11, wherein the at least one memory and the program code are configured to, with the processor, cause the apparatus to at least:
determine whether the death-based confirmation determination describes a positive determination; and
in response to determining that the death-based confirmation determination describes the positive determination, determine an inferior domain transmission rate determination based on the inferior domain death-based transmission rate.

13. The apparatus of claim 12, wherein the at least one memory and the program code are configured to, with the processor, cause the apparatus to at least:
in response to determining that the death-based confirmation determination describes the positive determination, cause performance of one or more resource allocation actions with respect to the inferior geographic domain based on the inferior domain transmission rate determination.

14. The apparatus of claim 9, wherein the case-based transmission rate trend describes a per-timestamp case-based transmission rate for each total time period timestamp of a plurality of total time period timestamps associated with the total time period.

15. The apparatus of claim 9, wherein the superior domain death-based transmission rate trend describes a per-timestamp superior domain death-based transmission rate for each prior time period timestamp of a plurality of prior time period timestamps associated with the prior time period in relation to the superior geographic domain.

16. The apparatus of claim 9, wherein the superior domain death-based transmission rate trend describes a per-timestamp superior domain death-based transmission rate for each prior time period timestamp of a plurality of prior time period timestamps associated with the prior time period in relation to the superior geographic domain.

17. A computer program product for performing cross-trend disease transmission predictive data analysis, the computer program product comprising at least one non-transitory computer-readable storage medium having computer-readable program code portions stored therein, the computer-readable program code portions configured to:
identify a case-based transmission rate trend for a superior geographic domain, wherein the case-based transmission is associated with a total time period comprising a prior time period and a current time period;
identify a superior domain death-based transmission rate trend for the superior geographic domain, wherein the superior domain death-based transmission rate trend is associated with the prior time period;
identify an inferior domain death-based transmission rate trend for an inferior geographic domain associated with the superior geographic domain, wherein the inferior domain death-based transmission rate trend is associated with the prior time period; and
cause the presentation of a predictive data analysis user interface, wherein the predictive data analysis user interface is configured to: (i) display a case-based transmission rate trend user interface element corresponding to the case-based transmission rate trend, a superior domain death-based transmission rate trend user interface element corresponding to the superior domain death-based transmission rate trend, and an inferior domain death-based transmission rate trend user interface element corresponding to the inferior domain death-based transmission rate trend, (ii) upon user interaction with a total time period segment of the case-based transmission rate trend user interface element that is associated with a total time period timestamp in the total time period, display a case-based confidence interval user interface element that is configured to describe a plurality of case-based transmission rate trend confidence interval values for the total time period timestamp, and (iii) upon user interaction with a prior time period segment of the case-based transmission rate user interface element that corresponds to a prior time period timestamp in the prior time period, display: (a) a superior domain death-based transmission rate user interface element that is configured to describe a superior domain death-based transmission rate for the superior geographic domain and the prior time period timestamp, and (b) an inferior domain death-based transmission rate user interface element that is configured to describe an inferior domain death-based transmission rate for the inferior geographic domain and the prior time period timestamp.

18. The computer program product of claim 17, wherein the predictive data analysis user interface is further configured to:
display a superior domain selection user interface element that is configured to enable selecting a selected superior geographic domain from a plurality of superior geographic domains, and
display an inferior domain selection user interface element that is configure to enable selecting a selected inferior geographic domain from a plurality of inferior geographic domains selected for the selected superior geographic domain.

19. The computer program product of claim 18, wherein the computer-readable program code portions are configured to:
generate an inferior domain confirmation determination for the inferior domain death-based transmission rate trend for the case-based transmission rate trend based on comparing a prior subset of the case-based transmission rate trend that is associated with the prior time period and the superior domain death-based transmission rate trend.

20. The computer program product of claim 19, wherein the computer-readable program code portions are configured to:
determine whether the death-based confirmation determination describes a positive determination; and
in response to determining that the death-based confirmation determination describes the positive determination, determine an inferior domain transmission rate determination based on the inferior domain death-based transmission rate.
